(19) United States
(12) Patent Application Publication
(10) Pub. No.: US 2025/0331895 A1
(43) Pub. Date: Oct. 30, 2025

(54) SKULL FIXATION DEVICES AND SYSTEMS

(71) Applicant: Rensselaer Polytechnic Institute, Troy, NY (US)

(72) Inventors: Katrina Ann Bliss, Huntingdon, PA (US); Grady Habicht, Hebron, CT (US); Taylor Lucille Keyt, Manchester, CT (US); Alexandra Danielle Bisaccia, Willston, VT (US); Sonya Elena Heldman, South Burlington, VT (US)

(73) Assignee: Rensselaer Polytechnic Institute, Troy, NY (US)

(21) Appl. No.: 18/860,798

(22) PCT Filed: Apr. 27, 2023
(86) PCT No.: PCT/US2023/020140

§ 371 Date: Oct. 28, 2024

Related U.S. Application Data
(60) Provisional application No. 63/335,484, filed on Apr. 27, 2022.

(51) Int. Cl. A61B 17/68; A61B 17/00; A61B 90/00

(52) U.S. Cl. CPC A61B 17/688; A61B 90/03; A61B 2017/00477; A61B 2017/00991; A61B 2017/681; A61B 2090/034

(57) ABSTRACT

A fixation device includes a base, a bracket positioned above the base, and a fastener connecting the bracket and the base. The base is configured to be attached to a first surface, such as a skull of a patient. The bracket includes an upper portion, a lower portion, and an intermediate portion connecting an end of the upper portion to an end of the lower portion. The lower portion is configured to be attached to a second surface, such as a bone flap removed from the skull of the patient. The fastener connects the upper portion of the bracket to the base. The fastener is configured to pass through at least one of an opening in the base and an opening in the upper portion of the bracket to incrementally adjust a distance between the base and the upper portion of the bracket.

### CROSS REFERENCE TO RELATED APPLICATIONS

This application claims the priority benefit of U.S. Provisional Patent Application No. 63/335,484, filed Apr. 27, 2022, which is incorporated by reference as if disclosed herein in its entirety.

### FIELD

The present technology relates generally to the field of bone fixation devices, and more particularly, to adjustable cranial fixation devices and system for use after craniectomy to improve patient outcomes and quality of life.

### BACKGROUND

Raised intracranial pressure is traditionally treated with decompressive craniectomy, which involves dural expansion by removing a large segment of bone. The goal of this procedure is to assist a patient through an intracranial pressure crisis by theoretically expanding the volume of the skull to infinity. The current surgical flow involves two invasive operations where a portion of the skull is first removed and cryopreserved and then returned to the remainder of the skull after several months. However, this current process of cryopreserving and then returning the portion of the skull results in high infection rates and patient mortality rates.

What is needed, therefore, is an improved surgical system that address at least the problems described above.

### SUMMARY

According to an embodiment of the present technology, a fixation device is provided. The fixation device includes a base having a first axial end and a second axial end as measured along a longitudinal axis of the fixation device. The base is configured to be attached to a first surface, such as a skull of a patient. A bracket is positioned above the base. The bracket includes an upper portion having a first axial end and a second axial end as measured along the longitudinal axis, a lower portion having a first axial end and a second axial end as measured along the longitudinal axis, and an intermediate portion connecting the second axial end of the upper portion to the first axial end of the lower portion. The lower portion is configured to be attached to a second surface, such as a bone flap removed from the skull of the patient. A fastener connects the upper portion of the bracket to the base. The fastener is configured to pass through at least one of an opening in the base and an opening in the upper portion of the bracket to incrementally adjust a distance between the base and the upper portion of the bracket.

In some embodiments, the intermediate portion of the bracket is substantially perpendicular to the longitudinal axis such that the bracket is substantially Z-shaped.

In some embodiments, when the distance between the base and the upper portion of the bracket is fully reduced by the fastener the first axial end of the lower portion of the bracket abuts the second axial end of the base such that the second surface is substantially flush with the first surface.

In some embodiments, the intermediate portion of the bracket is angled, and the second axial end of the base has an angled upper surface that corresponds to the angled intermediate portion of the bracket.

In some embodiments, when the distance between the base and the upper portion of the bracket is fully reduced by the fastener a bottom surface of the angled intermediate portion of the bracket abuts the angled upper surface of the second axial end of the base such that the second surface is substantially flush with the first surface.

In some embodiments, the fastener includes a cylindrical member having a threaded exterior circumferential surface thereon, and at least one of the opening in the base and the opening in the upper portion of the bracket is defined by a threaded interior circumferential surface configured to engage the threaded exterior circumferential surface of the fastener.

In some embodiments, the fastener is a telescoping fastener that includes an inner member and an outer member. The inner member is configured to be retained within a bore of the outer member, and the outer member is configured to pass through at least one of the opening in the base and the opening in the upper portion of the bracket.

In some embodiments, the inner member is cylindrical having a threaded exterior circumferential surface thereon, and the bore of the outer member is cylindrical and is defined by a threaded interior circumferential surface configured to engage the threaded exterior circumferential surface of the inner member.

In some embodiments, the outer member is cylindrical having a threaded exterior circumferential surface there, and at least one of the opening in the base and the opening in the upper portion of the bracket is defined by a threaded interior circumferential surface configured to engage the threaded exterior circumferential surface of the outer member.

In some embodiments, the base is attached to the first surface via fasteners and the lower portion of the bracket is attached to the second surface via fasteners.

In some embodiments, the first surface is a skull of a patient, and the second surface is a bone flap of the skull.

According to another embodiment of the present technology, an adjustable cranial fixation system is provided. The adjustable cranial fixation system includes a patient having a skull, a bone flap removed from the skull thereby forming a hole in the skull, and a plurality of fixation devices positioned around the perimeter of the hole in the skull. Each of the plurality of fixation devices includes a base, a bracket positioned above the base, and a fastener connecting the bracket to the base. The base has a first axial end and a second axial end as measured along a longitudinal axis of the fixation device. The base is attached to the skull adjacent the hole. The bracket includes an upper portion having a first axial end and a second axial end as measured along the longitudinal axis, a lower portion having a first axial end and a second axial end as measured along the longitudinal axis, and an intermediate portion connecting the second axial end of the upper portion to the first axial end of the lower portion. The lower portion is attached to the bone flap adjacent a perimeter edge thereof. The fastener connects the upper portion of the bracket to the base. The fastener is configured to pass through at least one of an opening in the base and an opening in the upper portion of the bracket to incrementally adjust a distance between the base and the upper portion of the bracket.

In some embodiments, the intermediate portion of the bracket is substantially perpendicular to the longitudinal axis such that the bracket is substantially Z-shaped.

In some embodiments, when the distance between the base and the upper portion of the bracket is fully reduced by the fastener the first axial end of the lower portion of the bracket abuts the second axial end of the base such that the bone flap is substantially flush with the skull.

In some embodiments, the intermediate portion of the bracket is angled, and the second axial end of the base has an angled upper surface that corresponds to the angled intermediate portion of the bracket.

In some embodiments, when the distance between the base and the upper portion of the bracket is fully reduced by the fastener a bottom surface of the angled intermediate portion of the bracket abuts the angled upper surface of the second axial end of the base such that the bone flap is substantially flush with the skull.

In some embodiments, the fastener includes a cylindrical member having a threaded exterior circumferential surface thereon, and at least one of the opening in the base and the opening in the upper portion of the bracket is defined by a threaded interior circumferential surface configured to engage the threaded exterior circumferential surface of the fastener.

In some embodiments, the fastener is a telescoping fastener that includes an inner member and an outer member. The inner member is configured to be retained within a bore of the outer member, and the outer member is configured to pass through at least one of the opening in the base and the opening in the upper portion of the bracket.

In some embodiments, the inner member is cylindrical having a threaded exterior circumferential surface thereon, and the bore of the outer member is cylindrical and is defined by a threaded interior circumferential surface configured to engage the threaded exterior circumferential surface of the inner member.

In some embodiments, the outer member is cylindrical having a threaded exterior circumferential surface there, and at least one of the opening in the base and the opening in the upper portion of the bracket is defined by a threaded interior circumferential surface configured to engage the threaded exterior circumferential surface of the outer member.

In some embodiments, the base is attached to the skull via fasteners and the lower portion of the bracket is attached to the bone flap via fasteners.

In some embodiments, the plurality of fixation devices are positioned such that the longitudinal axis of each fixation device is substantially aligned with a radial line of the hole in the skull.

Further objects, aspects, features, and embodiments of the present technology will be apparent from the drawing Figures and below description.

### BRIEF DESCRIPTION OF DRAWINGS

Some embodiments of the present technology are illustrated as an example and are not limited by the figures of the accompanying drawings, in which like references may indicate similar elements.

FIG. 1 is a side elevational view of a fixation device according to an embodiment of the present technology.

FIGS. 2A-2C are isometric views of the fixation device of FIG. 1 positioned at example incremental heights.

FIGS. 3A-3C are side elevational views of the fixation device of FIG. 1 attached to two surfaces and positioned at example incremental heights.

FIG. 4 is a side elevational view of a fixation device according to an embodiment of the present technology.

FIGS. 5A-5C are isometric views of the fixation device of FIG. 4 positioned at example incremental heights.

FIGS. 6A-6C are side elevational views of the fixation device of FIG. 4 attached to two surfaces and positioned at example incremental heights.

FIG. 7 is a side elevational view of a fixation device according to an embodiment of the present technology.

FIG. 8 is an isometric view of an adjustable cranial fixation system according to an embodiment of the present technology.

### DETAILED DESCRIPTION

Accordingly, exemplary embodiments of the present technology are directed to a fixation device and adjustable cranial fixation system used after craniectomy to improve patient outcomes and quality of life. Following a stroke or traumatic brain injury, some patients experience increased levels of intracranial pressure, resulting in a crisis situation and demanding immediate drug therapy, or, as a last result, neurosurgery. The current surgical flow involves two invasive surgeries where a portion of the skull, referred to herein as a bone flap, is first removed and cryopreserved and then returned to the remainder of the skull after several months. However, this second surgery results in high infection rates and patient mortality rates. Embodiments of the present technology allow for rigid fixation of the bone flap above the surgery site while facilitating incremental height decreases to return the bone flap to its original position flush with the remainder of the skull as the swollen brain reduces to its normal size. This removes the need for a second invasive surgery and instead requires only minimally invasive incisions to adjust the height of the device.

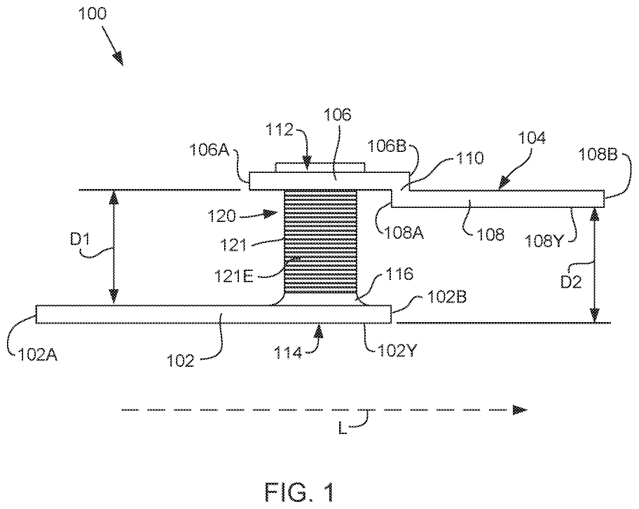

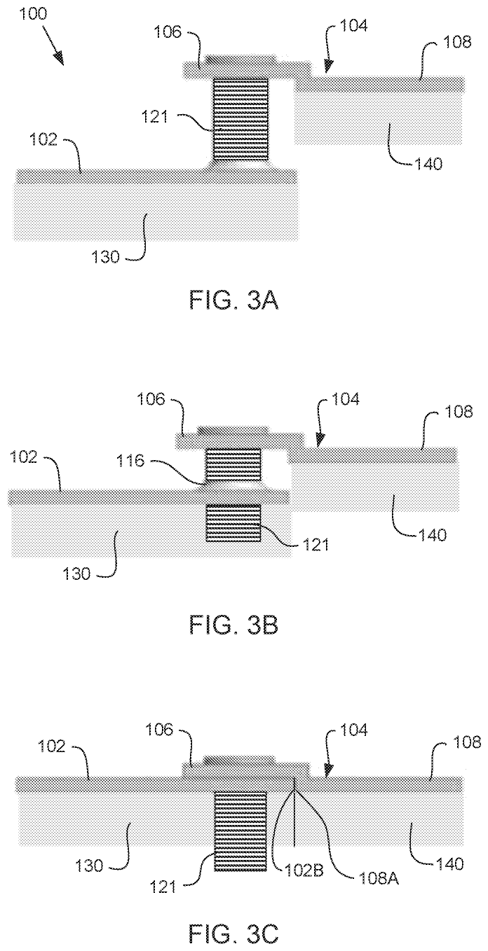

As shown in FIG. 1, a fixation device is generally designated by the numeral 100. The fixation device 100 includes a base 102 that has a first axial end 102A and a second axial end 102B as measured along a longitudinal axis L of the fixation device 100. The base 102 is configured to be attached to a first surface 130, as shown in FIGS. 3A-3C. In some embodiments, the first surface 130 is a skull of a patient who has undergone a craniectomy. The fixation device 100 includes a bracket 104 that is positioned above the base 102. The bracket 104 includes an upper portion 106, a lower portion 108, and an intermediate portion 110. The upper portion 106 has a first axial end 106A and a second axial end 106B as measured along the longitudinal axis L. The lower portion 108 has a first axial end 108A and a second axial end 108B as measured along the longitudinal axis L. The lower portion 108 is configured to be attached to a second surface 140, as shown in FIGS. 3A-3C. In some embodiments, the second surface 140 is a bone flap that was removed from the skull 130 of the patient during the craniectomy. The intermediate portion 110 connects the second axial end 106B of the upper portion 106 to the first axial end 108A of the lower portion 108. The fixation device 100 includes a fastener 120 that connects the upper portion 106 to the base 102. The fastener 120 is configured to pass through at least one of an opening 114 in the base 102 and an opening 112 in the upper portion 106 to adjust a distance DI between the base 102 and the upper portion 106. In some embodiments, the fixation device 100 is formed of a medical grade titanium. In some embodiments, the fixation device 100 is formed of a surgical stainless-steel material. In some embodiments, the fixation device 100 is formed of a high-performance semi-crystalline thermoplastic, such as polyetheretherketone. In some embodiments, the fixation device 100 is formed of a native cortical bone material. In some embodiments, the fixation device 100 is formed of a nonferrous metal material, such as titanium, aluminum, brass, copper, bronze, aluminum bronze alloy, etc.

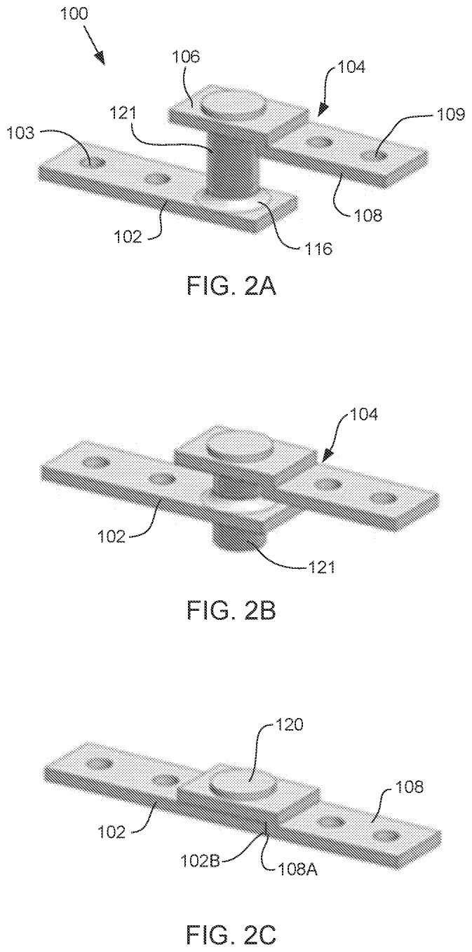

In some embodiments, the intermediate portion 110 is substantially perpendicular to the longitudinal axis L such that the bracket 104 is substantially Z-shaped, as shown in FIG. 1. In some embodiments, a distance D2 between a bottom surface 108Y of the lower portion 108 and a bottom surface 102Y of the base 102 is equal to the distance D1 such that when the distance D1 is fully reduced by the fastener 120 the second surface 140 is substantially flush with the first surface 130, as shown in FIGS. 3A-3C. In some embodiments, the intermediate portion 110 is positioned such that when the distance D1 is fully reduced by the fastener 120 the first axial end 108A of the lower portion 108 abuts the second axial end 102B of the base 102, as shown in FIGS. 2C and 3C.

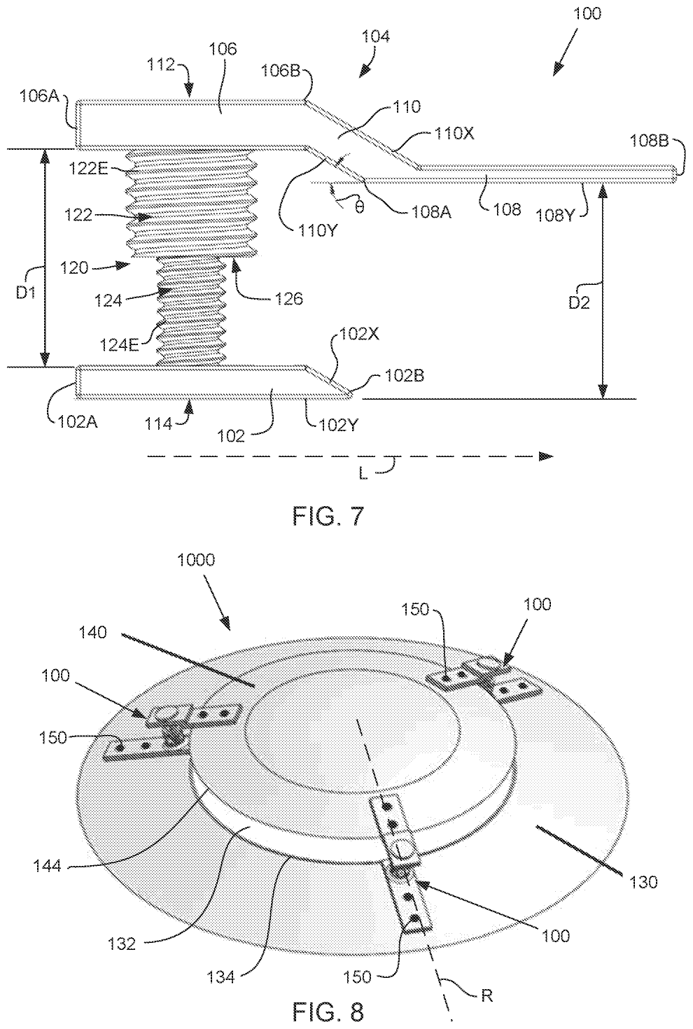

In some embodiments, the intermediate portion 110 is angled, and the second axial end 102B of the base 102 has an angled upper surface 102X that corresponds to an angled bottom surface 110Y of the intermediate portion 110, as shown in FIG. 7. In some embodiments, the intermediate portion 110 has an angled upper surface 110X that corresponds to the angled bottom surface 110Y. The angled bottom surface 110Y is angled away from the longitudinal axis L by an angle θ. In some embodiments, the angle θ is 30°. However, the present technology is not limited in this regard and contemplates embodiments where the angle θ is 15°, 45°, 60°, 75°, etc., or any angle within the range of 1° to 89°. In some embodiments, the distance D2 is equal to the distance D1 such that when the distance D1 is fully reduced by the fastener 120 the second surface 140 is substantially flush with the first surface 130. In some embodiments, the intermediate portion 110 is positioned such that when the distance D1 is fully reduced by the fastener 120 the angled bottom surface 110Y of the intermediate portion 110 abuts the angled upper surface 102X of the base 102.

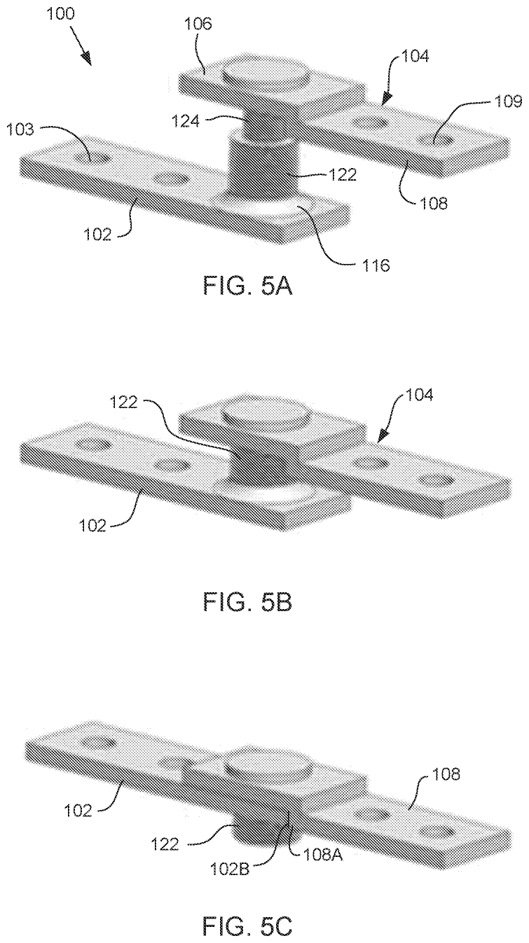

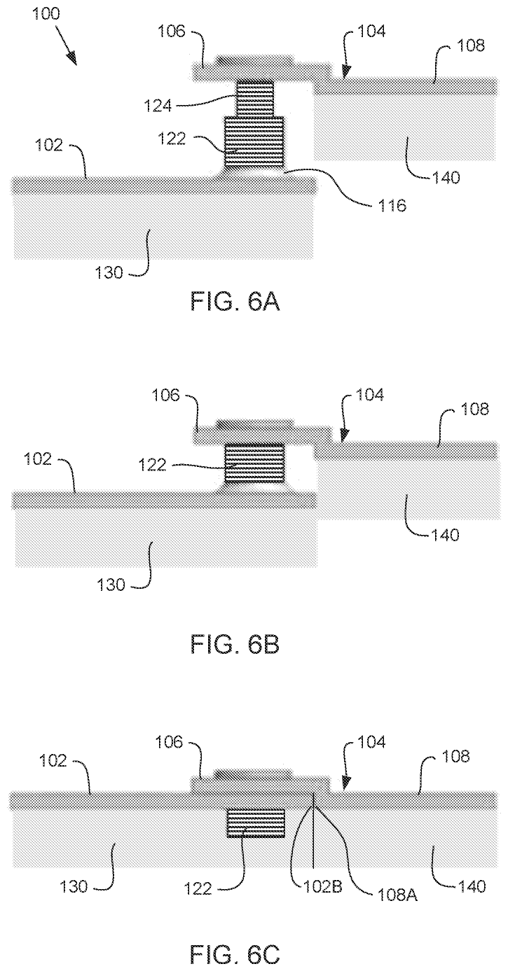

In some embodiments, the fastener 120 includes a cylindrical member 121 that has a threaded exterior circumferential surface 121E thereon, as shown in FIG. 1. At least one of the opening 114 in the base 102 and the opening 112 in the upper portion 106 is defined by a threaded interior circumferential surface that is configured to engage the threaded exterior circumferential surface 121E of the cylindrical member 121 such that rotation of the fastener 120 incrementally adjusts the distance D1. In some embodiments, at least one of the opening 114 in the base 102 and the opening 112 in the upper portion 106 is further defined by an apron 116 that serves as a washer to prevent the fastener 120 from loosening and/or to distribute the load from the fastener 120 over a larger area, as shown in FIGS. 1-6C.

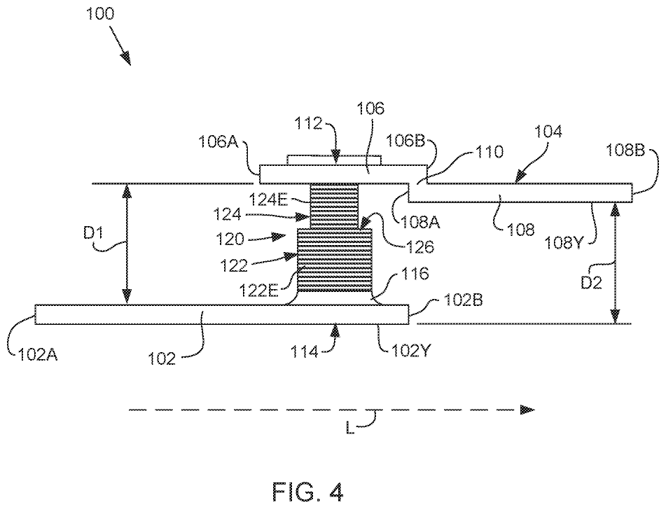

In some embodiments, the fastener 120 is a telescoping fastener that includes an inner member 124 and an outer member 122. The inner member 124 is configured to be retained within a bore 126 of the outer member 122, and the outer member 122 is configured to pass through at least one of the opening 114 in the base 102 and the opening 112 in the upper portion 106 after the inner member 124 is fully retained within the bore 126, as shown in FIGS. 4-6C.

As shown in FIGS. 4-7, in some embodiments, the inner member 124 is cylindrical and has a threaded exterior circumferential surface 124E thereon. The bore 126 of the outer member 122 is cylindrical and is defined by a threaded interior circumferential surface that is configured to engage the threaded exterior circumferential surface 124E of the inner member 124 to retain the inner member 124 within the bore 126. In some embodiments, the outer member 122 is cylindrical and has a threaded exterior circumferential surface 122E thereon. At least one of the opening 114 in the base 102 and the opening 112 in the upper portion 106 is defined by a threaded interior circumferential surface that is configured to engage the threaded exterior circumferential surface 122E of the outer member 122 such that rotation of the fastener 120 incrementally adjusts the distance D1. In some embodiments, the inner member 124 and the outer member 122 have equal lengths as measured perpendicular to the longitudinal axis L. In some embodiments, the length of the inner member 124 is less than the length of the outer member 122 as measured perpendicular to the longitudinal axis L. In some embodiments, the length of the inner member 124 is greater than the length of the outer member 122 as measured perpendicular to the longitudinal axis L. In some embodiments, the inner member 124 is attached to the upper portion 106 of the bracket 104, as shown in FIG. 4. In some embodiments, the inner member 124 is attached to the base 102, as shown in FIG. 7.

As shown in FIG. 8, an adjustable cranial fixation system is generally designated by the numeral 1000. The adjustable cranial fixation system 1000 includes a patient having a skull 130, and a bone flap 140 removed from the skull 130 thereby forming a hole 132 in the skull 130. A plurality of fixation devices 100, as described herein, are attached to the skull 130 and the bone flap 140 to rigidly fix the bone flap 140 above the hole 132 a height that is incrementally adjustable to return the bone flap 140 to its original position flush with the skull 130 as the patient's brains swelling reduces, as described above. In some embodiments, each fixation device 100 is attached to the skull 130 via fasteners 150 inserted through mounting holes 103 in the base 102, and each fixation device 100 is attached to the bone flap 140 via fasteners 150 inserted through mounting holes 109 in the lower portion 108 of the bracket 104. In some embodiments, each fixation device 100 is attached to the skull 130 and the bone flap 140 via an adhesive.

The plurality of fixation devices 100 are positioned around the hole 132 adjacent the perimeter edge 134 thereof. Each fixation device 100 is positioned such that the second axial end 102B of the base 102 is adjacent the hole 132, and preferably flush with the perimeter edge 134, and the first axial end 108A of the lower portion 108 is adjacent, and preferably flush with, the perimeter edge 144 of the bone flap 140. In some embodiments, each fixation device 100 is positioned such that the longitudinal axis L is substantially aligned with a radial line R of the hole 132 and/or the bone flap 140, as shown in FIG. 8. Although the embodiment shown in FIG. 8 shows three fixation devices 100 positioned around the hole 132 in substantially equal intervals, the present technology is not limited in this regard and contemplates embodiments having any number of fixation devices 100, such as one, two, four, five, six, seven, eight, etc., and being positioned around the hole 132 in equal or unequal intervals.

Accordingly, exemplary embodiments of the present technology are directed to a fixation device and adjustable cranial fixation system used after craniectomy to improve patient outcomes and quality of life. Embodiments of the present technology allow for rigid fixation of the bone flap above the surgery site while facilitating incremental height decreases to return the bone flap to its original position flush with the remainder of the skull as the swollen brain reduces to its normal size. This removes the need for a second invasive surgery and instead requires only minimally invasive incisions to adjust the height of the device.

As will be apparent to those skilled in the art, various modifications, adaptations, and variations of the foregoing specific disclosure can be made without departing from the scope of the technology claimed herein. The various features and elements of the technology described herein may be combined in a manner different than the specific examples described or claimed herein without departing from the scope of the technology. In other words, any element or feature may be combined with any other element or feature in different embodiments, unless there is an obvious or inherent incompatibility between the two, or it is specifically excluded.

References in the specification to “one embodiment,” “an embodiment,” etc., indicate that the embodiment described may include a particular aspect, feature, structure, or characteristic, but not every embodiment necessarily includes that aspect, feature, structure, or characteristic. Moreover, such phrases may, but do not necessarily, refer to the same embodiment referred to in other portions of the specification. Further, when a particular aspect, feature, structure, or characteristic is described in connection with an embodiment, it is within the knowledge of one skilled in the art to affect or connect such aspect, feature, structure, or characteristic with other embodiments, whether or not explicitly described.

The singular forms “a,” “an,” and “the” include plural reference unless the context clearly dictates otherwise. Thus, for example, a reference to “a plant” includes a plurality of such plants. It is further noted that the claims may be drafted to exclude any optional element. As such, this statement is intended to serve as antecedent basis for the use of exclusive terminology, such as “solely,” “only,” and the like, in connection with the recitation of claim elements or use of a “negative” limitation. The terms “preferably,” “preferred,” “prefer,” “optionally,” “may,” and similar terms are used to indicate that an item, condition, or step being referred to is an optional (not required) feature of the technology.

The term “and/or” means any one of the items, any combination of the items, or all of the items with which this term is associated. The phrase “one or more” is readily understood by one of skill in the art, particularly when read in context of its usage.

Each numerical or measured value in this specification is modified by the term “about.” The term “about” can refer to a variation of ±5%, ±10%, ±20%, or ±25% of the value specified. For example, “about 50” percent can in some embodiments carry a variation from 45 to 55 percent. For integer ranges, the term “about” can include one or two integers greater than and/or less than a recited integer at each end of the range. Unless indicated otherwise herein, the term “about” is intended to include values and ranges proximate to the recited range that are equivalent in terms of the functionality of the composition, or the embodiment.

As will be understood by one skilled in the art, for any and all purposes, particularly in terms of providing a written description, all ranges recited herein also encompass any and all possible sub-ranges and combinations of sub-ranges thereof, as well as the individual values making up the range, particularly integer values. A recited range (e.g., weight percents of carbon groups) includes each specific value, integer, decimal, or identity within the range. Any listed range can be easily recognized as sufficiently describing and enabling the same range being broken down into at least equal halves, thirds, quarters, fifths, or tenths. As a non-limiting example, each range discussed herein can be readily broken down into a lower third, middle third, and upper third, etc.

As will also be understood by one skilled in the art, all language such as “up to,” “at least,” “greater than,” “less than,” “more than,” “or more,” and the like, include the number recited and such terms refer to ranges that can be subsequently broken down into sub-ranges as discussed above. In the same manner, all ratios recited herein also include all sub-ratios falling within the broader ratio. Accordingly, specific values recited for radicals, substituents, and ranges, are for illustration only; they do not exclude other defined values or other values within defined ranges for radicals and substituents.

One skilled in the art will also readily recognize that where members are grouped together in a common manner, such as in a Markush group, the technology encompasses not only the entire group listed as a whole, but each member of the group individually and all possible subgroups of the main group. Additionally, for all purposes, the technology encompasses not only the main group, but also the main group absent one or more of the group members. The technology therefore envisages the explicit exclusion of any one or more of members of a recited group. Accordingly, provisos may apply to any of the disclosed categories or embodiments whereby any one or more of the recited elements, species, or embodiments, may be excluded from such categories or embodiments, for example, as used in an explicit negative limitation.

1. A fixation device comprising:
a base having a first axial end and a second axial end as measured along a longitudinal axis of the fixation device, the base configured to be attached to a first surface;
a bracket positioned above the base, the bracket comprising:
an upper portion having a first axial end and a second axial end as measured along the longitudinal axis;
a lower portion having a first axial end and a second axial end as measured along the longitudinal axis, the lower portion configured to be attached to a second surface; and
an intermediate portion connecting the second axial end of the upper portion to the first axial end of the lower portion; and
a fastener connecting the upper portion of the bracket to the base, the fastener configured to pass through at least one of an opening in the base and an opening in the upper portion of the bracket to incrementally adjust a distance between the base and the upper portion of the bracket.

2. The fixation device of claim 1, wherein the intermediate portion of the bracket is substantially perpendicular to the longitudinal axis such that the bracket is substantially Z-shaped.

3. The fixation device of claim 1, wherein when the distance between the base and the upper portion of the bracket is fully reduced by the fastener the first axial end of the lower portion of the bracket abuts the second axial end of the base such that the second surface is substantially flush with the first surface.

4. The fixation device of claim 1, wherein the intermediate portion of the bracket is angled, and the second axial end of the base has an angled upper surface that corresponds to the angled intermediate portion of the bracket.

5. The fixation device of claim 4, wherein when the distance between the base and the upper portion of the bracket is fully reduced by the fastener a bottom surface of the angled intermediate portion of the bracket abuts the angled upper surface of the second axial end of the base such that the second surface is substantially flush with the first surface.

6. The fixation device of claim 1, wherein the fastener comprises a cylindrical member having a threaded exterior circumferential surface thereon, and at least one of the opening in the base and the opening in the upper portion of the bracket is defined by a threaded interior circumferential surface configured to engage the threaded exterior circumferential surface of the fastener.

7. The fixation device of claim 1, wherein the fastener is a telescoping fastener comprising an inner member and an outer member, the inner member is configured to be retained within a bore of the outer member, and the outer member is configured to pass through at least one of the opening in the base and the opening in the upper portion of the bracket.

8. The fixation device of claim 7, wherein the inner member is cylindrical having a threaded exterior circumferential surface thereon, and the bore of the outer member is cylindrical and is defined by a threaded interior circumferential surface configured to engage the threaded exterior circumferential surface of the inner member.

9. The fixation device of claim 7, wherein the outer member is cylindrical having a threaded exterior circumferential surface there, and at least one of the opening in the base and the opening in the upper portion of the bracket is defined by a threaded interior circumferential surface configured to engage the threaded exterior circumferential surface of the outer member.

10. The fixation device of claim 1, wherein the base is attached to the first surface via fasteners and the lower portion of the bracket is attached to the second surface via fasteners.

11. The fixation device of claim 1, wherein the first surface is a skull of a patient, and the second surface is a bone flap of the skull.

12. An adjustable cranial fixation system comprising:
a patient having a skull;
a bone flap removed from the skull thereby forming a hole in the skull; and
a plurality of fixation devices positioned around the perimeter of the hole in the skull, each of the plurality of fixation devices comprising:
a base having a first axial end and a second axial end as measured along a longitudinal axis of the fixation device, the base is attached to the skull adjacent the hole;
a bracket positioned above the base, the bracket comprising:
an upper portion having a first axial end and a second axial end as measured along the longitudinal axis;
a lower portion having a first axial end and a second axial end as measured along the longitudinal axis, the lower portion is attached to the bone flap adjacent a perimeter edge thereof; and
an intermediate portion connecting the second axial end of the upper portion to the first axial end of the lower portion; and
a fastener connecting the upper portion of the bracket to the base, the fastener configured to pass through at least one of an opening in the base and an opening in the upper portion of the bracket to incrementally adjust a distance between the base and the upper portion of the bracket.

13. The system of claim 12, wherein the intermediate portion of the bracket is substantially perpendicular to the longitudinal axis such that the bracket is substantially Z-shaped.

14. The system of claim 12, wherein when the distance between the base and the upper portion of the bracket is fully reduced by the fastener the first axial end of the lower portion of the bracket abuts the second axial end of the base such that the bone flap is substantially flush with the skull.

15. The system of claim 12, wherein the intermediate portion of the bracket is angled, and the second axial end of the base has an angled upper surface that corresponds to the angled intermediate portion of the bracket.

16. The system of claim 15, wherein when the distance between the base and the upper portion of the bracket is fully reduced by the fastener a bottom surface of the angled intermediate portion of the bracket abuts the angled upper surface of the second axial end of the base such that the bone flap is substantially flush with the skull.

17. The system of claim 12, wherein the fastener comprises a cylindrical member having a threaded exterior circumferential surface thereon, and at least one of the opening in the base and the opening in the upper portion of the bracket is defined by a threaded interior circumferential surface configured to engage the threaded exterior circumferential surface of the fastener.

18. The system of claim 12, wherein the fastener is a telescoping fastener comprising an inner member and an outer member, the inner member is configured to be retained within a bore of the outer member, and the outer member is configured to pass through at least one of the opening in the base and the opening in the upper portion of the bracket.

19. The system of claim 18, wherein the inner member is cylindrical having a threaded exterior circumferential surface thereon, and the bore of the outer member is cylindrical and is defined by a threaded interior circumferential surface configured to engage the threaded exterior circumferential surface of the inner member.

20. The system of claim 18, wherein the outer member is cylindrical having a threaded exterior circumferential surface there, and at least one of the opening in the base and the opening in the upper portion of the bracket is defined by a threaded interior circumferential surface configured to engage the threaded exterior circumferential surface of the outer member.

21. The system of claim 12, wherein the base is attached to the skull via fasteners and the lower portion of the bracket is attached to the bone flap via fasteners.

22. The system of claim 12, wherein the plurality of fixation devices are positioned such that the longitudinal axis of each fixation device is substantially aligned with a radial line of the hole in the skull.
